(19) United States
(12) Patent Application Publication
(10) Pub. No.: US 2025/0281399 A1
(43) Pub. Date: Sep. 11, 2025

(54) A MUCOADHESIVE FILM COMPRISING A PHARMACEUTICALLY ACTIVE AGENT AND USES THEREOF

(71) Applicant: SHORT WAVE PHARMA INC., Toronto (CA)

(72) Inventors: Nadya LISOVODER, Zichron Yaakov (IL); Rivki STERN YOUDKEVICH, Ramat Hasharon (IL)

(73) Assignee: SHORT WAVE PHARMA INC., Toronto, ON (CA)

(21) Appl. No.: 18/861,988

(22) PCT Filed: May 1, 2023
(86) PCT No.: PCT/IL2023/050442

§ 371 Date: Oct. 31, 2024

Related U.S. Application Data
(60) Provisional application No. 63/337,118, filed on May 1, 2022.

(51) Int. Cl. A61K 9/00; A61K 31/4045; A61K 31/437; A61K 31/675; A61K 47/10; A61K 47/32

(52) U.S. Cl. CPC A61K 9/006; A61K 31/4045; A61K 31/437; A61K 31/675; A61K 47/10; A61K 47/32

(57) ABSTRACT

A mucoadhesive composition and method for the administration of the pharmaceutically effective agents is provided. Specifically, a mucoadhesive composition, composed of a mucoadhesive polymer and a plurality of active agents having different water solubility embedded therewithin; and use thereof such as for the treatment of a disease is provided.

### CROSS-REFERENCE TO RELATED APPLICATIONS

This application claims the benefit of priority of U.S. Provisional Patent Application No. 63/337,118, titled “MUCOADHESIVE FILM COMPRISING A PHARMACEUTICALLY ACTIVE AGENT AND USES THEREOF”, filed May 1, 2022, the contents of which are incorporated herein by reference in their entirety.

### FIELD OF THE INVENTION

The present invention, in some embodiments thereof, relates to a mucoadhesive composition comprising psilocybin, preparation and use thereof.

### BACKGROUND OF THE INVENTION

Psilocybin is a serotonin receptor agonist that occurs naturally in some mushroom species. Recent studies have assessed the therapeutic potential of psilocybin for various conditions, including anorexia, end-of-life anxiety, obsessive-compulsive disorder, and smoking and alcohol dependence, with promising preliminary results. Psilocybin (4-phosphoryloxy-N,N-dimethyltryptamine) is the major psychoactive alkaloid of some species of mushrooms distributed worldwide. Despite its experimental medical use in the 1960s, only very few pharmacological data about psilocybin were known until recently.

During the last few years, psilocybin and psilocin have gained therapeutic relevance but considerable physiological variability between individuals that can influence dose-response and toxicological profile has been reported. Psilocybin is rapidly dephosphorylated in the body to psilocin which acts as an agonist or partial agonist on several serotonin receptors.

There is an unmet need for pharmaceutical compositions and methods for transmucosal delivery of agents, e.g., psilocybin, such as in a sustained release manner. Further, for transmucosal delivery to the oral cavity, there is a need for compositions which are stable under dilution, shear flow and physiological conditions of the saliva fluids.

### SUMMARY OF THE INVENTION

The following embodiments and aspects thereof are described and illustrated in conjunction with systems, tools and methods which are meant to be exemplary and illustrative, not limiting in scope.

In one aspect, there is provided a pharmaceutical composition comprising: a mucoadhesive polymer (e.g. a biocompatible mucoadhesive polymer); a plasticizer; and a pharmaceutically active agent, wherein: the mucoadhesive polymer is a film forming polymer; the pharmaceutically active agent is (a) characterized by an aqueous solubility below 10 mg/ml, and (b) is a solid under normal conditions; a w/w concentration of the pharmaceutically active agent within the pharmaceutical composition is between 10 and 80%; the pharmaceutical composition is in a form of a film characterized by a thickness between 50 and 1000 um; and wherein the film is suitable for application on a mucous tissue of a subject.

In one embodiment, the mucoadhesive polymer is selected from a polyol, a polysaccharide modified or unmodified, including any combination or any copolymer thereof.

In one embodiment, polyol is or comprises polyvinyl alcohol (PVA).

In one embodiment, plasticizer comprises glycerol.

In one embodiment, the film is further in contact with a non-adhesive layer comprising a polymer selected from: a polyether, a polyester, a polydioxanone, a polyphosphoester, a polyurethane, and a polyamide or any mixture or a co-polymer thereof.

In one embodiment, the pharmaceutical composition is characterized by a prolonged release time of said pharmaceutically active agent.

In one embodiment, prolonged release time is greater by at least 10%, compared to a control.

In another aspect, there is a method for preventing or treating a medical condition in a subject, comprising administering the pharmaceutical composition of the invention, thereby preventing or treating said medical condition.

In one embodiment, said oral administration comprises buccal administration, sublingual administration or both.

In one embodiment, said medical condition comprises an eating disorder.

In one embodiment, said eating disorder comprises anorexia nervosa (AN).

In one embodiment, said medical condition comprises a neurodegenerative disease.

In another aspect, there is a method for delivering an active agent to a mucous tissue of a subject, comprising administering to a subject a composition comprising the active agent; a mucoadhesive polymer; and a plasticizer; wherein: the polymer is a film forming polymer; the active agent is (a) characterized by an aqueous solubility below 10 mg/ml, and (b) is a solid under normal conditions; a w/w concentration of the active agent within the composition is between 10 and 80%; the composition is in a form of a film characterized by a thickness between 50 and 1000 um; and wherein the film is suitable for application on a mucous tissue of the subject.

### BRIEF DESCRIPTION OF THE FIGURES

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIGS. 1A-1C are micrographs of the incorporated film, (1A) top layer, (1B) bottom layer, and (1C) cross section.

FIG. 2 is a graph presenting the swelling of the film in an artificial saliva fluid.

FIG. 3 is a graph presenting the release profile of Psilocybin and Norharmane, in a film comprising 40% drug load by weight.

FIG. 4 is a graph presenting the release profile of Psilocybin and Norharmane in a film comprising 20% drug load, and half a dose of a film comprising 40% drug load by weight.

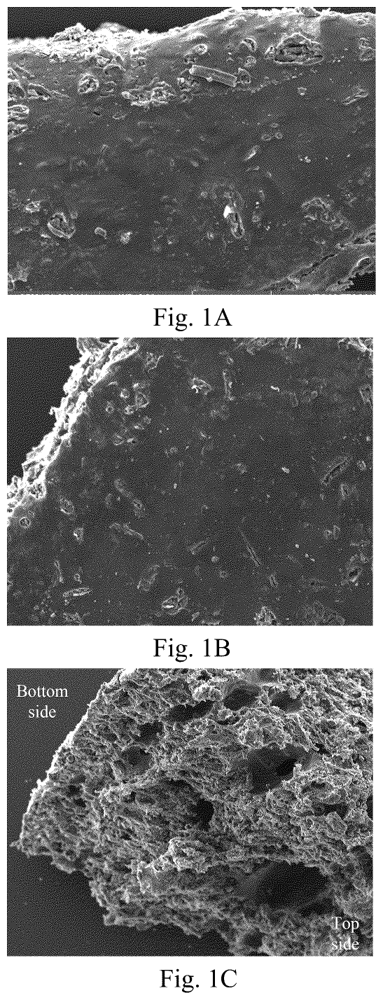

### DETAILED DESCRIPTION OF THE INVENTION

The present invention in some embodiments thereof refers to a pharmaceutical composition in a form of a film including a therapeutically effective amount of two or more active agents characterized by a low solubility in water (less than 10 mg in ml at about 25° C.), wherein the active agents comprise psilocybin (and/or psylocibin derivative) as the first active agent and beta carboline (and/or beta carboline derivative) as the second active agent, and wherein the first active agent has at least 5 times greater water solubility than the second active agent. Although the second active agent (beta carboline) has an extremely low water solubility, the inventors were able to find appropriate conditions for manufacturing a film (e.g., patch) having a sufficient loading of both psilocybin and beta carboline, while both active agents are homogeneously embedded within a water soluble mucoadhesive film forming polymer (e.g., PVA).

Furthermore, upon extensive experimentation the inventors found forming polymers which are particularly suitable for the formation of a thin mucoadhesive film which is: suitable for buccal application, capable for substantially releasing the active agents within a predefined time period, characterized by smooth appearance and sufficient elasticity for enhancing user compliance of the buccal patch.

In one aspect, the present invention relates to a pharmaceutical composition comprising a mucoadhesive polymer loaded with a pharmaceutically active agent, wherein the pharmaceutical composition is in a form of a solid film, suitable for buccal application (i.e., a buccal patch).

In another aspect, the present invention is related to a mucoadhesive and transdermal matrix comprising the pharmaceutically active agent dispersed therewithin. In some embodiments, the matrix being in a form of a solid film is for delivery of the pharmaceutically active agent via a mucosa. In some embodiments, the matrix is biodegradable or bioerodible. Further, the mucoadhesive matrix is configured to release the pharmaceutically active agent within a predetermined time period at the application site.

Moreover, the mucoadhesive matrix of the invention may be further shaped and sized in a form of a buccal patch, having numerous physico-chemical properties which are essential to assure high user compliance thereof. Such physico-chemical properties include inter alia a film thickness below 500 um (preferably below 300 um); transparency; mechanical stability and flexibility to assure ease of application; smooth and uniform outer surface; as well as additional properties such as high drug loading, and optimal disintegration rate at the application site.

In some embodiments, it is postulated that combining mucoadhesion with the advantages of sustained release rate of the active agent from the film of the invention, allows protecting the pharmaceutically active agent from chemical and enzymatic degradation, and improving its bioavailability, hence providing a powerful method for non-invasive drug delivery.

### Composition

In one aspect of the invention, there is provided a composition comprising an active agent (e.g., a pharmaceutically active agent, or a cosmetically active agent); a mucoadhesive polymer; and a plasticizer; wherein the mucoadhesive polymer is a film forming polymer; the active agent is a solid under normal conditions; a weight per weight (w/w) concentration of the active agent within the composition is between 10 and 80%; the composition is in a form of a film characterized by a thickness between 50 and 1000 um; and wherein the film is suitable for application on a mucous tissue of a subject. In some embodiments, the active agent is a pharmaceutically active agent.

In some embodiments, the composition is a pharmaceutical composition comprising inter alia a therapeutically effective amount of a pharmaceutically active agent distributed within a pharmaceutically acceptable mucoadhesive polymer. In some embodiments, the pharmaceutical composition comprises a mucoadhesive polymer; a plasticizer; and a pharmaceutically active agent, wherein: the mucoadhesive polymer is a film forming polymer; the pharmaceutically active agent is a solid under normal conditions; a w/w concentration of the pharmaceutically active agent within the pharmaceutical composition is between 10 and 80%; the pharmaceutical composition is in a form of a film characterized by a thickness between 50 and 1000 um; and wherein the film is suitable for application on a mucous tissue of a subject.

In some embodiments, the active agent is or comprises an alkaloid derived from a fungus. In some embodiments, the active agent is or comprises a psychoactive alkaloid. In some embodiments, the active agent (also used herein as the pharmaceutically active agent) is extracted from a fungus. In some embodiments, the active agent is in a purified form (e.g., being a pharmaceutically pure agent). In some embodiments, the active agent is in a form of a crude extract within the composition. In some embodiments, the active agent is a solid under normal conditions, comprising a temperature up to 30, or up to 40° C., and a normal atmospheric pressure, about 1 atm. In some embodiments, the active agent is an amorphous solid and/or a crystalline solid.

In some embodiments, a weight per weight (w/w) concentration of the active agent within the composition is between 10 and 80%, between 10 and 50%, between 20 and 80%, between 30 and 80%, between 30 and 50%, between 10 and 30%, between 10 and 40%, between 10 and 20%, between 10 and 30%, between 15 and 25%, between 10 and 25%, between 15 and 30%, including any range between.

In some embodiments, a weight per weight (w/w) concentration of the active agent by dry weight of the composition is between 10 and 50%, between 10 and 20%, between 10 and 30%, between 15 and 25%, between 10 and 25%, between 15 and 30%, between 10 and 40%, including any range between.

In some embodiments, a w/w concentration of the film forming polymer by dry weight of the composition is between 30 and 70%, between 30 and 60%, between 30 and 55%, between 40 and 60%, between 40 and 50%, including any range between.

In some embodiments, a w/w concentration of the plasticizer by dry weight composition is between 3 and 15%, between 3 and 10%, between 3 and 6%, between 3 and 9%, between 5 and 10%, including any range in between.

In some embodiments, a ratio between the film forming polymer and the plasticizer within the composition is between 10:1 and 1:1, between 5:1 and 1:1, between 5:1 and 2:1, between 8:1 and 1:1, between 8:1 and 2:1, between 6:1 and 4:1, including any range between. The term “dry weight” refers to a composition substantially devoid of water, i.e., wherein the water content is as disclosed herein (e.g., up to 10% by weight, or less).

In some embodiments, the composition of the invention (also used herein as the pharmaceutical composition) consists essentially of the mucoadhesive polymer, the plasticizer, and one or more active agents. In some embodiments, at least 90%, at least 92%, at least 95%, at least 97%, or between 90 and 99%, between 90 and 97%, between 90 and 95%, between 92 and 97%, between 95 and 99% by weight of the composition of the invention consists essentially of the mucoadhesive polymer, the plasticizer, and the active agent as disclosed herein.

In some embodiments, the composition of the invention consists essentially of the mucoadhesive polymer, the plasticizer, the first active agent and the second active agent. In some embodiments, at least 90%, at least 95%, including any range or value in between, of the composition of the invention consists of the mucoadhesive polymer, the plasticizer, the first active agent and the second active agent. In some embodiments, between 90 and 100%, between 90 and 95%, between 95 and 100%, between 92 and 98%, between 90 and 99%, between 90 and 97%, between 90 and 95%, between 92 and 97%, between 95 and 99% by weight of the composition of the invention including any range in between, consists of the mucoadhesive polymer, the plasticizer, the first active agent and the second active agent.

### Active Agent

In some embodiments, the active agent is or comprises a plurality of chemically distinct active agents. The term “plurality” encompasses 2, 3, 4, or 5 active agents, or alternatively not more than 3, not more than 4, or not more than 5 active agents. In some embodiments, the active agent is or comprises a first pharmaceutically active agent (also used herein as the first pharmaceutically active agent) and a second pharmaceutically active agent (also used herein as the second pharmaceutically active agent), wherein the first pharmaceutically active agent and the second pharmaceutically active agent have different water solubilities, and wherein the first pharmaceutically active agent has greater water solubility than the second pharmaceutically active agent. In some embodiments, the first pharmaceutically active agent has at least 2 times, at least 3 times, at least 5 times, at least 10 times, at least 20 times, at least 50 times greater water solubility than the second pharmaceutically active agent, including any range between, wherein the term “water solubility” is as disclosed below. In some embodiments, the first pharmaceutically active agent and the second pharmaceutically active agent have low water solubility.

As used herein, the term “water soluble” including any grammatical form thereof refers to the ability of a compound undergo dissolution in water (e.g., pure water devoid of an organic solvent and/or surfactant) at a temperature of between 20 and 27 or about 25° C., so as to form a homogenous solution, substantially devoid of a particulate matter (i.e., undissolved aggregates, or suspended solid particles of the compound). The presence of particles/aggregates in the solution can be determined by various methods, such as DLS.

Further, the term “water soluble” when referring to the first active agent being a psilocybin and/or a derivative thereof encompasses the water solubility of the zwitterionic form of the compound. Additionally, the term “water soluble” when referring to the second active agent being beta carboline and/or a derivative thereof, encompasses the water solubility of the free base form of the compound.

In some embodiments, the second pharmaceutically active agent has at least 2 times, at least 3 times, at least 5 times, at least 10 times, at least 20 times, at least 50 times greater ethanol solubility than the first pharmaceutically active agent, including any range between.

As used herein, the term “ethanol solubility” refers to the ability of a compound undergo dissolution in ethanol (e.g., pure ethanol having not more than 5% v/v of water) at a temperature of between 20 and 27 or about 25° C., so as to form a homogenous solution, substantially devoid of a particulate matter (i.e., undissolved aggregates, or suspended solid particles of the compound). The presence of particles/aggregates in the solution can be determined by various methods, such as DLS.

In some embodiments, the composition of the invention comprises a weight and/or molar excess of the second pharmaceutically active agent over the first pharmaceutically active agent. In some embodiments, a w/w ratio between the second active agent and the first active agent within the composition of the invention is between 1:1 and 10:1, between 1:1 and 5:1, between 2:1 and 5:1, between 1:1 and 4:1, between 3:2 and 4:1, between 2:1 and 4:1, including any range between.

In some embodiments, a w/w ratio between the second active agent and the first active agent within the composition of the invention is between 1:25 and 25:1, between 1:1 and 25:1, between 1:25 and 1:1, between 1:10 and 10:1, between 1:1 and 10:1, between 1:10 and 1:1, between 5:1 and 1:1, between 1:3 and 3:1, including any range between.

In some embodiments, at least 90%, at least 95%, at least 99%, between 90 and 100%, between 90 and 99%, between 95 and 99%, between 95 and 100% by total weight of the active agent within the composition of the invention consists of the first pharmaceutically active agent and the second pharmaceutically active agent, including any salt thereof. In some embodiments, the first pharmaceutically active agent and the second pharmaceutically active agent are the sole pharmaceutically active agents within the composition of the invention.

In some embodiments, the first active agent is characterized by a water solubility of at least 1 mg/ml, at least 2 mg/ml, at least 20 mg/ml and between 1 and 5 mg/ml, between 3 and 8 mg/ml, between 4 and 6 mg/ml, between 1 and 10 mg/ml, between 1 and 20 mg/ml, including any range between.

In some embodiments, the first active agent is or comprises psilocybin including any salt thereof, a psylocibin derivative including any salt thereof, or both. In some embodiments, the first active agent is or comprises psilocybin including any salt thereof and optionally further comprises psylocibin derivative.

In some embodiments, a psylocibin derivative comprises a structure represented by Formula 1, including any salt thereof:

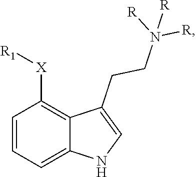

wherein X is O or OH; wherein R1 is H, POO⁻, POOH, or is absent; and wherein each R is independently hydrogen or methyl, with the proviso that the psylocibin derivative is not psylocibin. A skilled artisan will appreciate that in case all three R represent methyl groups, the amine attached to the Rs will be positively charged.

In some embodiments, the psylocibin derivative is an agonist of a serotonin receptor, such as 5-hydroxytryptamine receptor 2A (5-HT2A). In some embodiments, the psylocibin derivative modifies the biological activity of psilocybin. In some embodiments, the psilocybin derivative is represented by Formula 1 and is at least one of: (i) an alkaloid, (ii) is a psychoactive compound, and (iii) an agonist of a serotonin receptor. In some embodiments, the psylocibin derivative is psychoactive alkaloid. In some embodiments, the psylocibin derivative is psychoactive alkaloid extracted or isolated from a mushroom.

In some embodiments, at least one R group of the psilocybin derivative is methyl and the other R group(s) is/are hydrogen. In some embodiments, two R groups of the psilocybin derivative are methyl, and another R group is H. In some embodiments, all R groups of the psilocybin derivative are methyl.

In some embodiments, the psilocybin derivative is extracted from a fungus. In some embodiments, the psilocybin derivative is in a purified form (e.g., being a pharmaceutically pure agent). In some embodiments, the psilocybin derivative is in a form of a crude extract within the composition. In some embodiments, the psilocybin derivative is a solid under normal conditions, comprising a temperature between 10 and 30, or between 20 and 40° C., and a normal atmospheric pressure of about 1 atm. In some embodiments, the psilocybin derivative is an amorphous solid and/or a crystalline solid.

In some embodiments, the first active agent consists essentially of psylocibin, including any salt thereof.

In some embodiments, psylocibin is present within the composition of the invention in a form of a zwitterion:

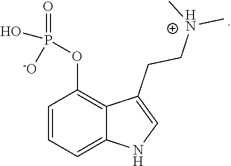

In some embodiments, psylocibin is in a form of a cation having a protonated (positively charged) amine and a protonated (uncharged) phosphate. In some embodiments, psylocibin or a derivative thereof is in a form of an anion having a free base (uncharged) amine and a protonated (negatively charged) phosphate. In some embodiments, psylocibin or a derivative thereof is uncharged, having a protonated (negatively charged) phosphate and a free base (uncharged) amine.

In some embodiments, between 50 and 100%, between 50 and 99%, between 50 and 97%, between 50 and 95%, between 50 and 90%, between 70 and 90%, between 80 and 90%, between 90 and 100%, between 80 and 99%, between 90 and 95%, between 90 and 99%, between 90 and 97% by weight of the first active agent within the composition is in a form of a zwitterion within the composition.

In some embodiments, at most 50%, at most 40%, at most 30%, at most 10%, at most 5%, at most 1%, at most 0.5%, or between 0.1% and 10%, between 0.1% and 5%, between 0.1% and 1% by weight of psylocibin, and/or a psylocibin derivative within the composition is in a form of which is not a zwitterion (such as a negatively charged compound, a positively charged compound or an uncharged compound).

In some embodiments, between 80 and 90%, between 80 and 100%, between 90 and 100%, between 80 and 99%, between 90 and 95%, between 90 and 99%, between 90 and 97% by weight of the first active agent is psylocibin, a psylocibin derivative or both.

In some embodiments, the first active agent is psylocibin. In some embodiments, between 50 and 100%, between 50 and 99%, between 50 and 97%, between 50 and 95%, between 50 and 90%, between 70 and 90%, between 80 and 90%, between 90 and 100%, between 80 and 99%, between 90 and 95%, between 90 and 99%, between 90 and 97% by weight of psylocibin is in a form of a zwitterion within the composition.

In some embodiments, at most 50%, at most 40%, at most 30%, at most 10%, at most 5%, at most 1%, at most 0.5%, or between 0.1% and 10%, between 0.1% and 5%, between 0.1% and 1% by weight of psylocibin within the composition is in a form of which is not a zwitterion.

In some embodiments, the weight percentage of the first active agent within the composition of the invention is between 2 and 20 wt %, between 2 and 15 wt %, between 3 and 15 wt %, between 3 and 8 wt %, between 3 and 10 wt %, between 4 and 6 wt %, between 4 and 15 wt %, between 5 and 15 wt %, between 8 and 13 wt %, including any range in between. In some embodiments, the weight percentage of the first active agent within the composition of the invention is between 8 and 13%, and wherein the first active agent is psylocibin, psylocibin salt or both.

In some embodiments, the second active agent has a water solubility of at most 1, at most 0.5 mg/ml, between 0.001 and 1, between 0.001 and 0.01, between 0.01 and 0.5, between 0.01 and 0.1, between 0.1 and 0.5, between 0.01 and 1, between 0.1 and 0.4 mg/ml, between 0.2 and 0.4 mg/ml, or about 0.3 mg/ml, including any range in between, wherein water solubility is as disclosed above.

In some embodiments, the second active agent is characterized by a solubility of at least 5, at least 10, at least 30, at least 50, at least 60, mg/ml or between 10 and 100, between 30 and 100, between 30 and 80, between 50 and 200, between 30 and 500, between 50 and 1000, mg/ml including any range between in ethanol.

In some embodiments, the second active agent is characterized by an ethanol solubility being at least 10 times, at least 100 times, at least 1000 times greater than the aqueous solubility of the second active agent, including any range between.

In some embodiments, the second active agent comprises a cyclic amine. In some embodiments, the second active agent comprises beta-carboline, a beta-carboline derivative, including any salt or any combination thereof.

In some embodiments, the second active agent is or comprises beta-carboline including any salt thereof and optionally further comprises a beta-carboline derivative.

In some embodiments, a beta-carboline derivative comprises a structure represented by Formula 2, including any salt thereof:

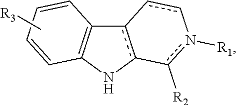

wherein R1 is absent, or represents hydrogen or methyl; wherein R2 is hydrogen or methyl; and wherein R3 is absent or represents one or more substituents each independently is selected from halogen, —OR′, —OH, —NO2, —CN, —CONH2, —CONR′2, —CNNR′2, —CSNR′2, —CONH—OH, —CONH—NH2, —NHCOR, —NHCSR, —NHCNR, —NC(═O)OR, —NC(═O)NR′, —NC(═S)OR′, —NC(═S)NR′, —SO2R′, —SOR′, —SR′, —SO2OR′, —SO2N(R′)2, —NHNR′2, —NNR′, —CO2H, —CO2R′, —OCOR, —OCOR′, —OC(═O)OR′, —OC(═O)NR′, —OC(═S)OR′, —OC(═S)NR′, or a combination thereof, wherein each R′ independently represents hydrogen, or methyl; and wherein a dash line represent a single or double bond, with the proviso that the beta-carboline derivative is devoid of beta carboline.

In some embodiments, the beta-carboline derivative is represented by Formula 2 and is at least one of: (i) an alkaloid, (ii) a MAO-A inhibitor, (iii) a convulsant alkaloid and/or (vi) an anxiogenic alkaloid.

In some embodiments, non-limiting examples of beta-carboline derivatives include but are not limited to: Tryptoline, Pinoline, Harman, Harmine, Harmaline, Tetrahydroharmine, and 9-Methyl-β-carboline, including any salt, or any combination thereof.

In some embodiments, between 80 and 90%, between 80 and 100%, between 90 and 100%, between 80 and 99%, between 90 and 95%, between 90 and 99%, between 90 and 97% by weight of the second active agent is beta-carboline, a beta-carboline derivative, or both.

In some embodiments, between 50 and 100%, between 50 and 99%, between 50 and 97%, between 50 and 95%, between 50 and 90%, between 70 and 90%, between 80 and 90%, between 90 and 100%, between 80 and 99%, between 90 and 95%, between 90 and 99%, between 90 and 97% by weight of the beta-carboline and/or a beta-carboline derivative within the composition is in a form of a free base within the composition.

In some embodiments, at most 50%, at most 40%, at most 30%, at most 10%, at most 5%, at most 1%, at most 0.5%, or between 0.1% and 10%, between 0.1% and 5%, between 0.1% and 1% by weight of the first active agent within the composition is in a form of which is not a free base (such as a positively charged amine salt).

In some embodiments, the second active agent is beta-carboline. In some embodiments, between 50 and 100%, between 50 and 99%, between 50 and 97%, between 50 and 95%, between 50 and 90%, between 70 and 90%, between 80 and 90%, between 90 and 100%, between 80 and 99%, between 90 and 95%, between 90 and 99%, between 90 and 97% by weight of beta-carboline is in a form of a free base within the composition.

In some embodiments, at most 50%, at most 40%, at most 30%, at most 10%, at most 5%, at most 1%, at most 0.5%, or between 0.1% and 10%, between 0.1% and 5%, between 0.1% and 1% by weight of beta-carboline within the composition is in a form which is not a free base (i.e. amine salt).

In some embodiments, the weight percentage of the second active agent within the composition of the invention is between 10 wt % and 50 wt %, between 10 wt % and 40 wt %, between 5 wt % and 30 wt %, between 10 wt % and 30 wt %, between 15 wt % and 40 wt %, between 25 wt % and 35 wt %, between 20 wt % and 40 wt %, including any range in between.

In some embodiments, a w/w ratio between the beta carboline related compound and the psylocibin within the composition of the invention is between 10:1 and 1:5, between 5:1 and 1:1, between 3:1 and 1:1, between 2:1 and 1:1, between 2:1 and 1:20, between 4:1 and 2:1, including any range between.

In some embodiments, the pharmaceutical composition is devoid of an additional therapeutically active ingredient which is not psylocibin, a derivative thereof, a beta-carboline, and/or a derivative thereof.

In some embodiments, the pharmaceutical composition of the invention comprises a therapeutically effective amount of the first pharmaceutically active agent and of the second pharmaceutically active agent. In some embodiments, the pharmaceutical composition of the invention comprises the first active agent at an amount sufficient for activating a serotonin receptor within the subject. In some embodiments, the pharmaceutical composition of the invention comprises the second active agent at an amount sufficient for inhibiting or reducing monoamine oxidase enzyme (MAOs) activity within the subject.

In some embodiments, the composition is in a form of a patch, and wherein a concentration (or therapeutically effective amount, or unit dose) of the pharmaceutically active within the patch is between about 1 and about 60 mg, between about 1 and about 10 mg, between about 1 and about 5 mg, between about 10 and about 60 mg, between about 10 and about 50 mg, between about 30 mg and about 50 mg, between about 30 mg and about 55 mg, including any range in between.

In some embodiments, a concentration (or therapeutically effective amount, or unit dose) of the first active agent within the patch is between 7 mg and 13 mg, between 7.5 mg and 12.5 mg, between 8 mg and 12 mg, between 9 mg and 11 mg, including any range in between. In some embodiments, a concentration (or therapeutically effective amount, or unit dose) of the second active agent within the patch is between 22 mg and 38 mg, between 23 mg and 36 mg, between 25 mg and 35 mg, between 27 mg and 32 mg, including any range in between.

As used herein, the term “therapeutically effective amount” refers to an amount effective, at dosages and for periods of time necessary, to achieve the desired therapeutic or prophylactic result in a subject. The exact dosage form and regimen would be determined by the physician according to the patient's condition.

### Film Former/Plasticizer

In some embodiments, the mucoadhesive polymer is a film forming polymer. In some embodiments, the mucoadhesive polymer is fully biocompatible. In some embodiments, the mucoadhesive polymer is at least partially biodegradable. In some embodiments, the mucoadhesive polymer is biodegradable and biocompatible. The terms “film forming polymer”, “mucoadhesive polymer” and “biocompatible mucoadhesive polymer” are used herein interchangeably.

As used herein, the term “biocompatible”, is intended to describe materials that are non-toxic to cells in vitro and upon administration in vivo, do not induce undesirable long-term effects.

As used herein, the term “biodegradable”, is intended to describe materials comprising covalent bonds that are degraded in vivo, wherein the degradation of the covalent bond occurs via hydrolysis. Hydrolysis can involve a direct reaction with an aqueous medium or can be catalyzed chemically or enzymatically. “Aqueous medium” refers to water, aqueous solutions, physiological media or biological fluids (e.g., body fluids), and other pharmaceutically acceptable media. Suitable hydrolysable covalent bonds are selected from the group containing: esters, amides, urethanes, carbamates, carbonates, ethers, azo linkages, anhydrides, thioesters, and combinations thereof. The term “biodegradable” also encompasses bioerodible material. The term “biodegradable” as used herein, refers to material and articles (e.g. compositions and/or films) comprising same, capable of undergoing at least partial degradation (or weight loss) under physiological conditions in vivo within a time period ranging between 1 hour (h) and 5 days (d), between 1 and 5 h, between 1 and 24 h, between 1 and 10 h, between 1 and 50 h, between 1 d and 5 d, between 1 and 2 d, including any range between.

In some embodiments, the plasticizer is or comprises a small molecule. In some embodiments, the plasticizer is compatible and/or miscible with the mucoadhesive polymer. As used herein the term “small molecule” means a molecule characterized by a molecular weight of up to 1000 Da.

In some embodiments, the mucoadhesive polymer is a water soluble, a water miscible and/or water dispersible polymer.

In some embodiments, the mucoadhesive polymer is at least partially cross-linked (e.g., via ionotropic cross-linking, complexation cross-linking, and/or covalent cross-linking).

In some embodiments, the mucoadhesive polymer is characterized by an average molecular weight ranging from 10,000 Da to 900,000 Da, from 10,000 Da to 100,000 Da, from 10,000 Da to 50,000 Da, from 50,000 Da to 100,000 Da, from 100,000 Da to 200,000 Da, from 200,000 Da to 300,000 Da, from 300,000 Da to 400,000 Da, from 400,000 Da to 500,000 Da, from 500,000 Da to 600,000 Da, from 600,000 Da to 900,000 Da, including any range or value therebetween.

In some embodiments, the mucoadhesive polymer is selected from: polyol (e.g., PVA), polysaccharide (alginate, chitosan, a gum), modified or un-modified starch, water-soluble starch alkyl ether (such as hydroxypropyl methylcellulose (HPMC)), polyacrylate (modified or un-modified, such as polyacrylic acid), polyalkycyanoacrylate, polylactide, polylactide-polyglycolide copolymer, polycaprolactone, dextran, albumin, gelatin, collagen, cyclodextrin, sodium crosscarmelose, polyvinylpyrolidone and polyethyleneimine, including any co-polymer or any combination thereof. In some embodiments, the mucoadhesive polymer comprises PVA, or HPMC including any copolymer or any combination thereof.

In some embodiments, the PVA is characterized by an average molecular weight ranging between 10,000 Da to 30,000 Da, between 13,000 Da and 23,000 Da, between 10,000 Da and 25,000 Da, between 10,000 Da and 23,000 Da, between 15,000 Da and 20,000 Da, between 10,000 Da and 25,000 Da, between 13,000 Da and 30,000 Da, including any range in between.

In some embodiments, the PVA is characterized by an average molecular weight ranging between 13,000 Da and 23,000 Da.

In some embodiments, the PVA is characterized by hydrolyzation degree (from the PVAcetate) of between 80% and 100%, between 80% and 95%, between 80% and 90%, between 85% and 90%, between 85 and 95%, between 85 and 99%, including any range in between.

In some embodiments, the plasticizer enhances elasticity of the composition and/or of the mucoadhesive polymer. In some embodiments, the plasticizer reduces the elastic modulus of the composition by at least 10%, at least 12%, at least 14%, at least 15%, at least 17%, at least 20%, including any range between, compared to the elastic modulus of the pristine mucoadhesive polymer.

The plasticizer provides a certain degree of flexibility, stretch ability or elasticity to the composition (or film), so as to result in a deformable, pliable, or stretchable composition which can be applied to the mucous tissue of the subject (e.g., via an applicator). In some embodiments, the plasticizer enhances elasticity of the film of the invention so as to improve compliance thereof.

In some embodiments, the plasticizer reduces a glass transition temperature and/or crystallinity of the mucoadhesive polymer, thus increasing (e.g., by 10%, 30%, 50% 100%, or more) break resistance thereof, compared to a pristine mucoadhesive polymer devoid of the plasticizer.

In some embodiments, the plasticizer is selected from glycerol, triethyl citrate (TEC), Myvacet (Myv, an acetoglyceride-based emulsifier), sorbitan mono-oleate or monostearate (SMO, SMS), a glycol (e.g., propylene glycol (PG), ethylene glycol, etc.), glyceryl mono-oleate (GMO), and glyceryl mono-stearate (GMS) or any combination thereof. In some embodiments, the plasticizer is a surface-active agent (e.g., an emulsifier). The term “surfactant” is well understood by a skilled artisan, as being related inter alia to an amphiphilic agent reducing the surface tension of two immiscible liquids.

In some embodiments, the plasticizer is or comprises glycerol.

In some embodiments, the composition of the invention is in a form of a film.

In some embodiments, the film is in a solid state. In some embodiments, the film is substantially devoid of a semi-solid or semi liquid (e.g., a gel). In some embodiments, the film is an adhesive film. In some embodiments, the film is a bioadhesive film.

In some embodiments, the film comprises a polymeric matrix. In some embodiments, the matrix comprises a plurality of polymeric layers. In some embodiments, the plurality of polymeric layers comprises or are mucoadhesive polymeric layers. In some embodiments, the matrix comprises layers having the same or different compositions. In some embodiments, the matrix comprises a plurality of distinct layers. In some embodiments, the matrix is a multi-layer matrix. In some embodiments, multi-layer matrix comprises a mucoadhesive layer and an additional layer. In some embodiments, the film comprises a polymeric adhesive matrix. In some embodiments, the polymeric adhesive matrix is a mucoadhesive matrix. In some embodiments, the polymeric adhesive matrix is a biodegradable and/or biocompatible mucoadhesive matrix.

As used herein, the term “matrix” refers to one or more layers of polymeric chains. In some embodiments, the polymeric chains are randomly distributed within the matrix. In some embodiments, the randomly distributed polymeric chains within the matrix, form a three-dimensional mesh structure comprising a void space between the chains. In some embodiments, the randomly distributed polymeric chains form a plurality of pores (or void space). In some embodiments, the matrix is substantially devoid of aligned or oriented polymeric chains. In some embodiments, the matrix is substantially devoid of polymeric chains aligned or oriented in a specific direction. Matrix may further include any materials incorporated within and/or interposed between the layers.

In some embodiments, at least 70%, at least 80%, at least 90%, at least 95%, at least 99%, at least 99.9% by weight of the matrix consists of the mucoadhesive polymer. In some embodiments, the film of the invention further comprises a carrier in a form of nanoparticles and/or lipid-based particles (e.g., liposomes). In some embodiments, carrier is in a form of a vesicle such as that carried materials (therapeutic agent or diagnostic agent) are inside an internal core. In some embodiments, at least one carrier is a lipid-based particle. In another embodiment, at least one carrier is a micelle.

In some embodiments, the carrier is in a form of a particle (e.g., a liposome, a micelle, a vesicle, etc.). In some embodiments, the particles are distributed within the mucoadhesive polymer bulk (or matrix) substantially uniformly in a single particle form or in the form of clusters of particles. In some embodiments, the particles are distributed within the mucoadhesive multilayered matrix.

In some embodiments, the carrier of the invention encapsulates the pharmaceutically active agent. In some embodiments, the carrier of the invention is formulated for mucosal and/or transdermal delivery of one or more pharmaceutically active agents.

In some embodiments, the film comprises a first adhesive surface and a second surface. In some embodiments, the second surface is substantially non-adhesive. In some embodiments, the second surface is devoid of adhesiveness. As used herein the term “adhesive” refers to adhesiveness of the film or a part thereof to a biological tissue.

As used herein, the term “biological tissue” refers to any surface comprising cells and/or biological molecules (e.g., proteins, polysaccharides, lipids, nucleic acids).

In some embodiments, the film is a single layered film. In some embodiments, the film is a multi-layered film. In some embodiments, the multi-layered film comprises a layer comprising the composition of the invention in contact with a subsequent layer being devoid of an active agent. In some embodiments, the layer is in a form of a continuous layer. In some embodiments, the film comprises a bottom layer and a top layer. In some embodiments, the bottom layer and the top layer are biodegradable. In some embodiments, any one of the layers, the bottom layer and the top layer comprise a biodegradable polymer.

In some embodiments, the film (e.g., in a form of one or more layers) comprises the bottom layer and the top layer and further comprises an intermediate layer, wherein the intermediate layer is substantially devoid of the active agent. In some embodiments, the intermediate layer comprises a polymeric material or polymeric matrix. In some embodiments, the intermediate layer is characterized by a degradation (or erosion) rate being less than a degradation (or erosion) rate of any one of the bottom layer and the top layer. In some embodiments, the intermediate layer is configured to prolong the release time of the active agent.

The term “continuous layer” or the term “layer” refers to a substantially homogeneous substance of substantially uniform-thickness which maintains its physico-chemical properties (e.g., mechanical strength, elasticity, Young's modulus, chemical composition, water content) with the entire dimensions (lengths and width dimensions) thereof. In some embodiments, each layer has a different physical structure and/or a different chemical composition. In some embodiments, each layer has the same physical structure and/or the same chemical composition. In some embodiments, the term “layer”, refers to a mucoadhesive polymer layer.

In some embodiments, the film is at least partially biodegradable and/or bioerodible. In some embodiments, the film of the invention is substantially biodegradable and/or bioerodible. In some embodiments, the degradation rate of the film is predetermined by the chemical composition, average molecular weight (MW), and/or the structure (e.g., branched versus linear polymer; cross-linking degree; block co-polymer versus graft co-polymer) of the mucoadhesive polymer. In some embodiments, the composition of the invention is biocompatible.

One skilled in the art will appreciate, that by controlling or adjusting the degradation rate of the mucoadhesive polymer and/or of the film comprising thereof, the adhesive strength and/or release rate of the pharmaceutically active agent may be affected. In some embodiments, the degradation rate of the mucoadhesive polymer and/or the article comprising thereof, predetermines (e.g., increases or decreases by at least 10%) the release rate of the pharmaceutically active agent from the composition of the invention. In some embodiments, the degradation rate of the mucoadhesive polymer predetermines (e.g., increases or decreases by at least 10%) the adhesive strength of the composition comprising of the invention.

In some embodiments, a w/w concentration of the first pharmaceutically active agent within the film is between 0.1 and 30%, is between 0.1 and 10%, is between 5 and 20%, is between 5 and 10%, is between 10 and 15%, is between 15 and 20%, is between 20 and 30%, is between 10 and 30%, is between 5 and 15%, between 5 and 25%, including any range therebetween.

In some embodiments, a w/w concentration of the second pharmaceutically active agent within the film of the invention is between 10 and 50%, between 10 and 40%, between 10 and 30%, between 15 and 25%, between 15 and 35%, between 20 and 40%, between 25 and 35%, between, including any range therebetween.

In some embodiments, the film of the invention has a water content of at most 10%, at most 8%, at most 7.5%, or at most 7% by weight of the film, including any range in between. In some embodiments, the water content of the film of the invention is between 0.1% and 10%, between 0.5% and 10%, between 0.5% and 8%, between 0.1% and 8%, between 2% and 8%, between 2% and 7%, between 3% and 8%, between 4% and 8% by weight including any range in between. The inventors presume that a higher water content than 10% w/w may result in a sticky film, thus reducing user compliance.

In some embodiments, the composition of the invention is characterized by a thickness of between 1 μm and 1 mm thick a width of between 1 mm and 5 cm, and a length of between 1 mm and 5 cm.

In some embodiments, the composition of the invention is characterized by a thickness from 1 to 10 μm, from 5 to 200 μm, from 5 to 500 μm from 10 to 20 μm, from 20 to 30 μm, from 30 to 40 μm, from 40 to 50 μm, from 10 to 500 μm, from 10 to 100 μm, from 40 to 500 μm, from 40 to 100 μm, from 40 to 300 μm, from 50 to 100 μm, from 100 to 150 μm, from 100 to 500 μm, from 200 to 400 μm, from 150 to 200 μm, from 200 to 300 μm, from 300 to 400 μm, from 400 to 500 μm, from 500 to 600 μm, from 600 to 700 μm, from 700 to 800 μm, from 800 to 900 μm, from 900 to 1000 μm, from 1000 to 2000 μm, from 2000 to 3000 μm, from 3000 to 5000 μm, from 100 to 5000 μm, including any range or value therebetween.

In some embodiments, the composition of the invention is characterized by a thickness of between 5 to 500 μm, between 5 to 400 μm between 5 to 300 μm, between 5 to 200 μm, between 10 to 250 μm, between 50 to 250 μm, between 5 to 250 μm, between 50 to 200 μm, between 30 to 200 μm, between 25 to 225 μm, between 70 to 150 μm, between 50 to 150 μm, including any range or value therebetween.

In some embodiments, the film of the invention is between 5 and 300 μm, or between 5 and 250 μm thick.

In some embodiments, the terms “thick” or “thickness” including any grammatical form thereof, refer to an average dry thickness. As used herein, the term “dry thickness” refers to the thickness of the dried film.

In some embodiments, the film of the invention is characterized by a width of between 1 and 50 mm, between 1 and 10 mm, between 1 and 20 mm, between 10 and 30 mm, between 10 and 40 mm, between 5 and 15 mm, between 15 and 25, including any range in between.

In some embodiments, the film of the invention is characterized by a length of between 1 and 50 mm, between 1 and 10 mm, between 1 and 20 mm, between 10 and 30 mm, between 10 and 40 mm, between 5 and 15 mm, between 15 and 25, including any range in between.

In some embodiments, the film of the invention is characterized by a density of between about 50 and 200 mg/cm³, between about 80 and 150 mg/cm³, between about 100 and 130 mg/cm³, between about 105 and 130 mg/cm³, between about 100 and 200 mg/cm³, between about 110 and 125 mg/cm³, between about 100 and 150 mg/cm³, between about 110 and 180 mg/cm³, including any range or value in between.

In some embodiments, the film of the invention is characterized by an area density of between about 0.05 and 0.15 g/cm², between about 0.05 and 0.13 g/cm², between about 0.07 and 0.13 g/cm², between about 0.05 and 0.11 g/cm², between about 0.07 and 0.11 g/cm², between about 0.05 and 0.09 g/cm², between about 0.07 and 0.09 g/cm², between about 0.06 and 0.10 g/cm², including any range or value in between.

In some embodiments, the film of the invention is characterized by an adhesion strength of at least 30 KPa, at least 40 KPa, at least 50 KPa, at least 60 KPa, at least 70 KPa, at least 80 KPa, at least 90 KPa, at least 100 KPa, at least 200 KPa, including any range therebetween, wherein the adhesion strength is measured according to an adhesion test.

In some embodiments, the film of the invention is characterized by a smooth surface, determined by visual observation. In some embodiments, the smooth surface is devoid of defect and/or bumps, as determined by visual observation.

In some embodiments, the film of the invention is characterized by a tensile strength sufficient for application thereof to the target site.

In some embodiments, the composition of the invention provides for a flexible patch-like substrate configured for external application at a body site. In some embodiments, the composition is for the treatment or for the delivery of a pharmaceutically active agent to the subject.

In some embodiments, the composition of the invention is for application on top of a biological tissue. In some embodiments, the biological tissue comprises a mucous tissue, a dermal tissue, or both.

In some embodiments, the film is shapeable. In some embodiments, at least one dimension of the film is variable, e.g., by applying stress. In some embodiments, the film is shapeable along at least one dimension, e.g., a length dimension, a width dimension, a radial dimension, a diagonal dimension, and the like. In some embodiments, the film may be shaped and/or elongated, e.g., by a user and/or a medical practitioner, to become elongated, wider, increased in diameter, and/or a combination thereof.

In some embodiments, the film is foldable. In some embodiments, the film is flexible. In some embodiments, the film is characterized by elasticity. In some embodiments, the film is characterized by elasticity and/or foldability sufficient for application of the article on one or more region of the tissue (e.g., mucous or dermal tissue) of the subject.

In some embodiments, the film further comprises an additive (e.g., a coloring agent, a taste agent, etc.). In some embodiments, other additives which can be beneficially incorporated into the composition of the invention include both natural and/or synthetic polymeric (macro-biomolecules, for example, proteins, enzymes) and non-polymeric (small molecule therapeutics) natural or synthetic agents.

In some embodiments, the film of the invention is configured to substantially release the pharmaceutically active agent (e.g., the first pharmaceutically and the second pharmaceutically active agents). In some embodiments, the composition is configured to release the pharmaceutically active agent due to at least partially degradation and/or erosion of the film. In some embodiments, the film of the invention is characterized by a gradual release profile of the second pharmaceutically active agent (e.g., into the tissue and/or into a location adjacent to the application site). In some embodiments, the first pharmaceutically and the second pharmaceutically active agent are characterized by different release profiles.

In some embodiments, the composition of the invention is configured to substantially release at least 50%, at least 60%, at least 70%, at least 80%, at least 90%, at least 93%, at least 95%, at least 97%, at least 99% by weight of first pharmaceutically active agent including any range between, wherein first pharmaceutically active agent refers to the initial amount of the first pharmaceutically active agent within the composition.

In some embodiments, the composition of the invention is configured to substantially release the first pharmaceutically active agent from the composition within a time period ranging between 10 min and 2 days, between 10 and 60 min, between 10 and 30 min, between 20 and 40 min, between 30 and 60 min, between 1 h and 2 h, between 1 and 5 h, between 10 min and 5 h, between 1 and 24 h, including any range between.

In some embodiments, the composition is characterized by a prolonged release time of the first pharmaceutically active agent, compared to a control. In some embodiments, prolonged comprise at least 20%, at least 50%, at least 60%, at least 70%, at least 80%, at least 90%, at least 95%, at least 97%, at least 99%, at least 200%, at least 300%, at least 400%, at least 500% greater release time, compared to a control.

As used herein, the term “control” refers to pristine pharmaceutically active agent (e.g. in a form of a crystalline or amorphous solid) and is not embedded within a film forming polymer.

In some embodiments, the composition of the invention is configured to substantially release at least 30%, at least 40%, at least 50%, at least 60%, at least 70%, at least 80%, at least 90%, at least 93%, at least 95% by weight of second pharmaceutically active agent including any range between, wherein second pharmaceutically active agent refers to the initial amount of the second pharmaceutically active agent within the composition.

In some embodiments, the composition of the invention is configured to substantially release the second pharmaceutically active agent from the composition within a time period ranging between 10 min and 4 days, between 10 and 60 min, between 1 h and 2 h, between 10 min and 4 h, between 1 and 5 h, between 10 min and 5 h, between 10 min and 24 h, including any range between.

In some embodiments, the composition is characterized by a prolonged release time of the second pharmaceutically active agent, compared to a control. In some embodiments, prolonged comprise at least 20%, at least 50%, at least 60%, at least 70%, at least 80%, at least 90%, at least 95%, at least 97%, at least 99%, at least 200%, at least 300%, at least 400%, at least 500% greater release time, compared to a control.

In some embodiments, the composition is characterized by a prolonged release time of the second pharmaceutically active agent, compared to the first pharmaceutically active agent. In some embodiments, prolonged comprise at least 20%, at least 50%, at least 60%, at least 70%, at least 80%, at least 90%, at least 95%, at least 97%, at least 99%, at least 200%, at least 300%, at least 400%, at least 500% greater release time, compared to the first pharmaceutically active agent.

In some embodiments, the film is further in contact with an additional layer. In some embodiments, the additional layer is in a form of capping and/or protecting layer. In some embodiments, the additional layer is in a barrier layer, configured for preventing undesired adhesion of the film. In some embodiments, the additional layer is configured for preventing adsorption of particles (e.g., dust, moisture, or any other contamination) on top of the film. In some embodiments, the additional layer comprises a packaging material. In some embodiments, the additional layer comprises a polymeric material. In some embodiments, the additional layer comprises a thermoplastic polymer. In some embodiments, the additional layer is a non-adhesive layer.

In some embodiments, the non-adhesive layer comprises a polymer comprising a polyether, a polyester, a polydioxanone, a polyphosphoester, a polyurethane, and a polyamide or any mixture or a co-polymer thereof.

In some embodiments, the non-adhesive layer comprises a polymer selected from the group comprising: polytetrafluoroethylene, a fluorinated polyolefin, polyvinyl fluoride, polyethylene terephthalate (PET), polycyclohexylenedimethylene terephthalate, polycyclohexylenedimethylene terephthalate (PCTG), polyethylene naphthalate (PEN), polytrimethylene terephthalate (PTT), polybutylene terephthalate (PBT), a polyether, polyethyleneglycol (PEG), polypropylene (PP), polyethylene (PE), polycarbonate, polycaprolactone (PCL), polylactic acid (PLA), polylactic-co-glycolic acid (PLGA), polyhydroxyethyl methacrylate (polyHEMA), and polyurethane, including any combination or a copolymer thereof.

In some embodiments, the composition of the invention is an article. In some embodiments, the article is a medical device. In some embodiments, the article is characterized by a controlled release of the pharmaceutically active agent. In some embodiments, the article is in a form of a patch. In some embodiment, the article is a film.

In some embodiments, the composition of the invention is characterized by a mechanical stability. As used herein the term “stability” refers to the capability of the film of the invention to maintain its structural and/or mechanical integrity. In some embodiments, the stable film or article substantially maintains its adhesiveness to the target site. In some embodiments, the stable film or article is applicable to a target site of a subject. In some embodiments, the stable film or article maintains its structural and/or mechanical integrity at the target site for at least 1 h, at least 2 h, at least 3 h, at least 5 h, including any range between. In some embodiments, the target site is a biological tissue.

In some embodiments, the film or article of the invention is referred to as stable, when at least 90%, at least 95%, at least 99%, by weight of the drug load remains compared to the initial loading, including any value and range therebetween.

In some embodiments, the film or article of the invention is referred to as stable, when at least 90%, at least 95%, at least 99%, by weight of the drug load remains compared to the initial loading, for a period of at least 1 w, at least 1 m, at least 2 m, at least 4 m, at least 6 m, at least 9 m, at least 1 year, including any value and range therebetween.

In some embodiments, the stable film or article is rigid under operable conditions. In some embodiments, the stable film is inert to the operable conditions. The operable conditions may be referred to physiological conditions, such as physiological conditions of a mucosal and/or dermal tissue (e.g., oral and/or nasal cavity) such as pH (e.g., between 5.5 and 8.5), moisture, enzymatic species, and temperature (e.g., between 35 and 38° C.) or any combination thereof).

In some embodiments, the article comprises an adhesive outer layer. In some embodiments, the adhesive outer layer is configured to adhere to a tissue of a subject (e.g., at an application site). In some embodiments, the outer layer has adhesiveness sufficient so as to retain the article at the application site for at least 1 h, at least 2 h, at least 3 h, at least 5 h, including any range between.

In some embodiments, the composition of the invention can be used for delivery of the pharmaceutically active agent to an area of the body having a mucous membrane, such as, but not limited to, the oral cavity. In some embodiments, the composition can be used for transdermal absorption or transdermal delivery of an active agent to a subject in need thereof. For example, the delivery vehicle can be designed for use in oral, buccal, nasal, rectal and vaginal routes for both systemic and local effects. In some embodiments, the composition can be used for transdermal absorption or transdermal delivery of a pharmaceutically effective amount of an active agent to the subject.

### Uses

According to another aspect of some embodiments of the present invention there is provided a method for administering an active agent to a biological tissue of a subject, comprising contacting the film of the invention with the biological tissue of the subject, thereby administering the pharmaceutically active agent to the subject. In some embodiments, the film of the invention comprises a pharmaceutically effective amount of the pharmaceutically active agent. In some embodiments, the film of the invention is for administering a therapeutically effective amount of an active agent to a biological tissue of a subject.

In some embodiments, the biological tissue is as described herein. In some embodiments, the biological tissue is a moist tissue. In some embodiments, the biological tissue is as described herein. In some embodiments, the biological tissue is a substantially dry tissue (e.g., dermal tissue). In some embodiments, the biological tissue is a mucous and/or dermal tissue.

In some embodiments, contacting comprises providing the film and applying the film to a target site of the subject. In some embodiments, applying comprises contacting the adhesive surface with the target site (moist or dry) on or within the biological tissue or organ of the subject. In some embodiments, upon contacting the film with the biological tissue, the adhesive surface of the film faces or is bound to the biological tissue. In some embodiments, applying comprises pressing the film towards the biological tissue, so as to induce adhesion of the film thereto.

In some embodiments, the film or patch of the invention is a medical device. In some embodiments, the medical device is for use in the field of drug delivery. In some embodiments, the medical device is for administering a pharmaceutically effective amount of the active agent of the invention to the subject (e.g., to a target site on or within the mucous tissue). In some embodiments, administering comprises local administration and/or systemic administration. In some embodiments, the active agent is as described herein above.

In some embodiments, the method is for topically administering the active agent. In some embodiments, the method is for mucosal or transdermal administration (e.g., oral or nasal administration) of the active agent.

In some embodiments, administering is selected from the group consisting of oral administration, vaginal administration, rectal administration, ocular administration, nasal administration, topical administration and dermal administration, or any combination thereof. In some embodiments, oral administration comprises buccal administration, sublingual administration or both. In some embodiments, the method is for delivery of the active agent into a mucous or dermal tissue. In some embodiments, the method is for controlled delivery and/or release of the active agent into a mucous or dermal tissue of the subject.

In some embodiments, the method is for transmucosal and/or transdermal administration of the active agent. In some embodiments, the method is for sustained administration of the active agent. In some embodiments, the method is for sustained release of the active agent to the target site. In some embodiments, the method is for sustained release of the active agent to a biological tissue of the subject. In some embodiments, the biological tissue comprises a mucous tissue, a dermal tissue, a muscle tissue, and a urinary bladder tissue or any combination thereof.

In some embodiments, the method is for sustained release of the active agent into the mucous tissue and blood circulation. In some embodiments, administration and/or release comprises a pharmaceutically effective amount of the active agent. It is postulated that the active agent has an improved absorption via a mucosal tissue.

According to another aspect of some embodiments of the present invention there is provided a method for preventing or treating a medical condition, comprising administering the composition (or film) of the invention to a subject, thereby preventing or treating the medical condition, wherein the composition comprises a pharmaceutically effective amount of an active agent. In some embodiments, administering comprises contacting the composition (or film) with a biological tissue of the subject, as described herein. In some embodiments, administering is by buccal administration.

In some embodiments, the method is for reducing and/or ameliorating a symptom associated with the medical condition within the subject.

In some embodiments, administering comprises oral or nasal administration. In some embodiments, administering comprises topical administration. In some embodiments, administering comprises dermal administration. In some embodiments, administering comprises transdermal administration. Other administration routes are as described hereinabove.

In some embodiments, the herein disclosed composition (or film) is for treating a medical condition or ameliorating a symptom associated therewith, wherein the medical condition comprises an eating disorder, chronic pain and neurodegenerative diseases. In some embodiments, the eating disorder is selected from the group consisting of anorexia nervosa, bulimia nervosa, binge eating disorder, pica, rumination disorder, and avoidant/restrictive food intake disorder (ARFID) or a combination thereof.

In some embodiments, the herein disclosed composition (or film) is for treating a medical condition or ameliorating a symptom associated therewith, wherein the medical condition comprises chronic pain (fibromyalgia, Complex regional pain syndrome (CRPS)), a neurodegenerative disease (e.g., Alzheimer's disease, dementia, dementia with Lewy bodies, Parkinson's disease, Huntington's disease, etc.).

In some embodiments, there is a method for treating anorexia nervosa, comprising administering the composition (or film) of the invention to a subject, thereby preventing or treating anorexia nervosa.

In some embodiments, there is a method for treating a condition selected from chronic pain (fibromyalgia, Complex regional pain syndrome (CRPS)), and neurodegenerative disease, comprising administering the composition (or film) of the invention to a subject. In some embodiments, the method of treating chronic pain (fibromyalgia), and/or neurodegenerative disease comprises administering to the subject the film of the invention.

In some embodiments, the film (e.g., a single-layered or a multi-layered matrix) disclosed herein is a medical device. In some embodiments, the medical device is an implantable medical device.

According to an aspect of embodiments of the invention there is provided a medicament comprising one or more films (or articles) disclosed herein and a pharmaceutically acceptable carrier.

As used herein, the term “subject” refers to any animal (e.g., a mammal), including, but not limited to, humans, non-human primates, rodents, and the like, to which the compositions and methods of the present invention are administered. In some embodiments, the terms “subject” and “patient” are used interchangeably herein in reference to a human subject. In other embodiments, the terms “subject” and “patient” are used interchangeably herein in reference to a non-human subject.

As used herein the term “reducing” in any grammatical form thereof comprises at least 5%, 10%, 15%, 20%, 25%, 30%, 35%, 40%, 45%, 50%, 60%, 70%, 80%, 100%, 200%, 500%, 1000% or higher reduction of one or more value or parameter, including any range therebetween.

As used herein the term “enhancing” or the term “increasing” in any grammatical form thereof comprises at least 5%, 10%, 15%, 20%, 25%, 30%, 35%, 40%, 45%, 50%, 60%, 70%, 80%, 100%, 200%, 500%, 1000% or higher enhancement of one or more value or parameter, including any range therebetween.

According to one aspect, there is provided a method for manufacturing the film of the invention (or the pharmaceutical composition of the invention in a form of a film) comprising (i) providing the mucoadhesive polymer, the pharmaceutical active agent, the plasticizer and mixing the entire constituents with a solvent (either consecutively or simultaneously), thereby obtaining an aqueous composition; (ii) shaping the aqueous composition according to predefined dimensions, thereby obtaining a wet film; and (iii) drying the wet film, thereby obtaining the film of the invention (or the pharmaceutical composition of the invention in a form of a film).

In some embodiments, step (i) comprises mixing mucoadhesive polymer and optionally the plasticizer with the solvent, thereby obtaining a solution; and adding the pharmaceutical active agent (e.g. in a form of a solid, or in a form of a liquid composition such as a solution, a suspension or a dispersion) to the solution.

In some embodiments, mixing is performed at a temperature of between 20° C. and 80° C., between 20° C. and 30° C., between 20° C. and 70° C., between 30° C. and 60° C., between 20° C. and 60° C., including any range in between. In some embodiments, step (i) comprises mixing mucoadhesive polymer and optionally the plasticizer with the solvent at a temperature of between 20° C. and 80° C., between 20° C. and 30° C., between 20° C. and 70° C., between 30° C. and 60° C., between 20° C. and 60° C., including any range in between, thereby obtaining a solution; and adding the pharmaceutical active agent to the solution, thereby obtaining the aqueous composition.

In some embodiments, the mixing further comprises a cooling step prior to the adding step; wherein the solution is cooled to a temperature between 20° C. and 30° C., or about 25° C.

In some embodiments, the solvent consists essentially of water, or an aqueous buffer, or an aqueous salt solution. In some embodiments, the solvent is an aqueous solvent being substantially devoid of an organic solvent (i.e. the organic solvent content of the solvent is not more than 5%, not more than 1%, not more than 0.5% by volume of the solvent). In some embodiments, the solvent is a water ethanol mixture, wherein the ethanol content within the solvent is between 30 and 90%, including any range between.

In some embodiments, step (i) comprises providing the pharmaceutical active agent in a solid form, or in a form of a liquid composition such as a solution, a suspension or a dispersion.

In some embodiments, the aqueous composition of step (i) is characterized by a viscosity of up to 12,000 cp, up to 11,000 cp, up to 10,000 cp, including any range or value in between. In some embodiments, the wet composition is characterized by a viscosity of between 2,000 cp and 12,000 cp, between 2,000 cp and 10,000 cp, between 8,000 cp and 12,000 cp, between 8,000 cp and 10,000 cp, between 5,000 cp and 10,000 cp, or between 6,000 cp and 12,000, including any range in between.

In some embodiments, the aqueous composition of step (i) is characterized by a w/w concentration of between 10% and 30% of PVA, between 5% and 10% of glycerol, between of 10% and 30% of Norharmane, between 5% and 13% of Psilocybin and between 30 and 70% of water.

In some embodiments, shaping is by a method selected from but not limited to: casting, solvent casting and molding.

In some embodiments, the wet film is characterized by a water content of at least 20%, at least 30%, at least 40% and between 20% and 70%, between 30% and 70%, between 40% and 60%, between 30% and 60%, or between 30% and 50%, including any range in between.

In some embodiments, the drying step is by a method selected from but not limited to: vacuum drying, air-drying and oven drying.

### General:

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure. Specifically, the term “consisting essentially of” encompasses that at least 90%, at least 95%, at least 97%, at least 99%, at least 99.5% by weight of the composition, article or material, or any of the constituents thereof (e.g. mucoadhesive polymer, pharmaceutical active agent, etc.) solely includes the compounds or materials listed herein.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict. The words “further” and “optionally” may be used interchangeably.

As used herein, the singular form “a”, “an” and “the” include plural references unless the context clearly dictates otherwise. For example, the term “a compound” or “at least one compound” may include a plurality of compounds, including mixtures thereof.

As used herein, the term “substantially” is at least 80%, at least 90%, at least 92%, at least 95%, at least 97%, at least 98%, at least 99% by weight of the composition including any range or value therebetween.

As used herein, the term “water miscible” refers to the ability of two liquids to form a homogenous mixture (a single phase) at a range of temperature (e.g., between 20 and 60° C.), pressure and the liquid ratios.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treatment” or “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

While the present invention has been particularly described, a person skilled in the art will appreciate that many variations and modifications can be made. Therefore, the invention is not to be construed as restricted to the particularly described embodiments, and the scope and concept of the invention will be more readily understood by reference to the claims, which follow.

The following Examples are representative of techniques employed by the inventors in carrying out aspects of the present invention. It should be appreciated that while these techniques are exemplary of embodiments for the practice of the invention, those of skill in the art, in light of the present disclosure, will recognize that numerous modifications can be made without departing from the intended scope of the invention.

### EXAMPLES

The inventors successfully manufactured mucoadhesive transparent films comprising up to about 40% by weight of an active agent (e.g., Norharmane) and characterized by sufficient flexibility and elasticity. The inventors observed that implementing higher drug loading (about 50% or more), a film with inferior physical-chemical properties has been obtained (e.g., a brittle film with rough outer surface).

Exemplary film has been composed of PVA and glycerol in about 3:1 weight ratio, respectively (between about 5-500 um thick). Additionally, HPMC/glycerol-based film with a similar drug load has been manufactured (about 50-400 um thick). Uniform drug load and good absorption were seen in film thickness ranging between 5 and 200 um.

To decrease the disintegration time of 1 cm² of polymer film, the inventors added a plasticizer to the polymeric solution and films were cast. The addition of plasticizer decreases the disintegration time (Table 1).

TABLE 1
the effect the plasticizer has on the HPMC film formation.

| | | | | | Disinte- |
|---|---|---|---|---|---|
| | | | | | gration |
| | | | | | time of |
| | | | Film | Mass of 1 | 1 cm² |
| | Plasti- | | thickness | cm² of | of polymer |
| | ciser | Film | (μm) | polymer | film |
| Polymer | (0.1%) | behavior | (n = 5) | film (mg) | (min:sec) |
| HPMC 15 | N/A | Easy to | 101.9 | 9.4 | 2:54 |
| LV 100 | | peel, | | | |
| mg/mL | | foldable | | | |
| | Glycerol | Easy to | 108.8 | 9.9 | 3:16 |
| | | peel, | | | |
| | | foldable | | | |
| | Triacetin | Easy to | 106.6 | 9.8 | 3:12 |
| | | peel, | | | |
| | | foldable | | | |
| | Propylene | Easy to | 114 | 11.5 | 3:14 |
| | Glycol | peel, | | | |
| | | foldable | | | |

Furthermore, the inventor observed that films made from different polymers, in different solvents had similar film quality, whereas the thickness differed but was in the desired range between 5 and 250 um. Exemplary films were prepared from (i) PVA:glycerol using water as the solvent or alternatively (ii) HPMC/glycerol in the using ethanol water (80/20) solution. These results demonstrate that a diversity of polymers can be used to form the film of the invention.

TABLE 2
HPMC and PVA film properties.

| | | Film | |
|---|---|---|---|
| Excipients | | thickness | |
| conc. (mg/mL) | Solvent | (μm) | Observation |
| HPMC 606 (150) + | Ethanol/water | 63.47 ± 2.62 | |
| glycerol (30) | (80/20) | | |
| HPMC 606 (150) + | | 65.90 ± 3.08 | Film quality |
| propylene glycol (30) | | | similar, |
| HPMC 606 (150) + | | 55.70 ± 2.65 | foldable but |
| polyethylene | | | slightly hard |
| glycol (30) | | | |
| PVA (100) + | water | 35.23 ± 2.19 | Film soft and |
| glycerol (230) | | | flexible |

### Example 1

### Film and Suspension Stability

### Suspension/Film Preparation

PVA and glycerol solution in water at a PVA:glycerol ratio of 3:1 was prepared by string the constituents at 50° C. until homogenization. The solution was cooled to room temperature and two different concentrations (20% or 40% w/w) of Norharmane and Psilocybin were added at a ratio of 3:1 respectively. Homogeneous suspensions were obtained after 1 h of stirring at room temperature.

Films were prepared using a 3 mm gap applicator and dried at room temperature. The drug loading was examined in the dry film, formed from the 20% drug load suspension and found to be 40%. Advantages physical and chemical properties were observed for films with a thickness of between 5 and 250 um, such as uniform drug loading and uniform thickness.

### Stability

Suspension stability was examined under storage at 2 to 8° C., and determined by HPLC, before injection the samples were stirred, and the drug loading within the film was measured at different periods of time. The suspension was stable even after 8 days.

For stability assessments, the film with a drug load of 40 mg was cut into 1 cm² squares, sealed in foiled bags and stored at 25° C. with a relative humidity of 60% or at 5° C. The drug load was determined by HPLC at t=0, after three months (n), six months, and nine months no major difference was observed, results are summarized in table 3,

TABLE 3
Drug load of Norharmane and Psilocybin after three, six and nine months.

| | HPLC results | | | | | | | |
|---|---|---|---|---|---|---|---|---|
| | 25° C./60% RH | | | | 2-8° C. | | | |
| | Norhamane | Psilocybin | Total | | Norhamane | Psilocybin | Total | |
| | Drug | Drug | Drug | | Drug | Drug | Drug | |
| Time | load | load | load | Purity | load | load | load | Purity |
| point | tested (%) | tested (%) | (%) | (%) | tested (%) | tested (%) | (%) | (%) |
| t0 | 28.7 ± | 9.7 ± | 38.3 ± | 99.88 ± | 28.7 ± | 9.7 ± | 38.3 ± | 99.88 ± |
| | 1.2 | 0.4 | 1.6 | 0.004 | 1.2 | 0.4 | 1.6 | 0.004 |
| t2 w | 27.1 ± | 9.3 ± | 36.3 ± | 99.85 ± | 27.2 ± | 9.3 ± | 36.4 ± | 99.85 ± |
| | 1.1 | 0.2 | 0.3 | 0.00 | 0.1 | 0.04 | 0.1 | 0.01 |
| t4 w | 26.2 ± | 9.1 ± | 35.3 ± | 99.86 ± | 26.3 ± | 9.2 ± | 35.4 ± | 99.86 ± |
| | 0.3 | 0.3 | 0.6 | 0.001 | 0.3 | 0.03 | 0.3 | 0.001 |
| t3 M | 28.4 ± | 9.7 ± | 38.1 ± | 99.86 ± | 28.5 ± | 9.7 ± | 38.1 ± | 99.87 ± |
| | 0.2 | 0.01 | 0.2 | 0.02 | 0.06 | 0.01 | 0.06 | 0.02 |
| t6 M | 28.4 ± | 10.1 ± | 38.5 ± | 99.78 ± | 28.6 ± | 10.0 ± | 38.7 ± | 99.78 ± |
| | 0.1 | 0.3 | 0.2 | 0.01 | 0.2 | 0.04 | 0.3 | 0.004 |
| t9 M | 27.5 ± | 9.1 ± | 36.6 ± | 99.69 ± | 28.2 ± | 9.5 ± | 37.7 ± | 99.72 ± |
| | 0.9 | 0.3 | 1.2 | 0.05 | 0.0 | 0.1 | 0.1 | 0.01 |

### Film Characterization

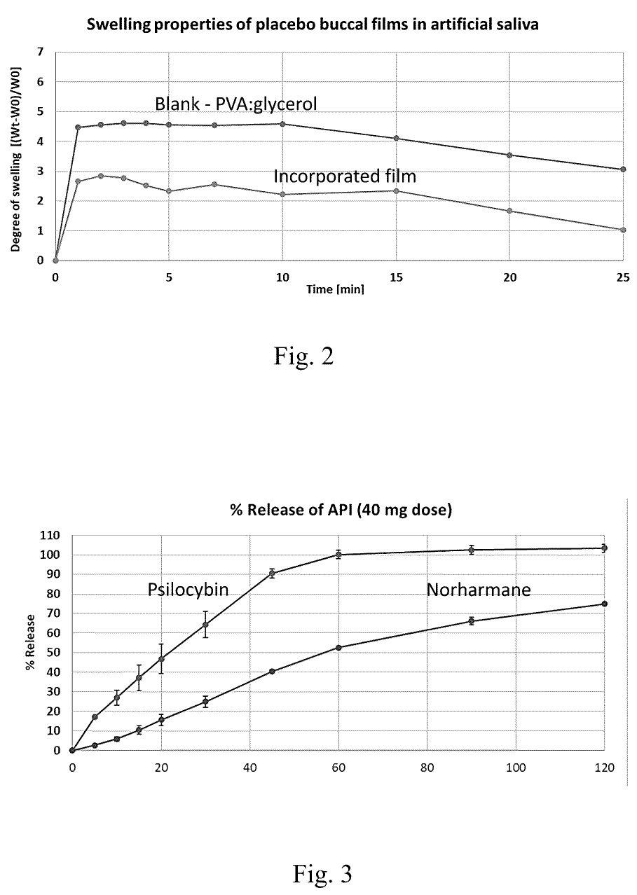

The swelling properties of the film (comprising Norharmane and Psilocybin) were tested in artificial saliva fluid compared to a PVA-glycerine film devoid of active agents (FIG. 2). The film exhibits faster swelling onset and lower degree of swelling compared to a PVA-glycerine film.

### Example 2

### Release Profile of Norharmane and Psilocybin

The release profile of Psilocybin and Norharmane from an exemplary composition (patch) of the invention was measured by exposing the patch to 900 ml of simulated saliva fluid (SSF, pH 6.8). A sample was taken every 5, 10, 15, 20, 30, 45, 60, and 120 min, and the concentration of Psilocybin and Norharmane was measured in the dissolution of the patch, by high pressure liquid chromatography (HPLC).

A patch comprising a total drug load of 40 mg (comprising 30 mg Norharmane and 10 mg of psilocybin) was measured, all of the Psilocybin was released after 1 hour. Whereas Norharmane demonstrated a slower release rate, where only 75% was released after 2 h (FIG. 3), using HPLC.

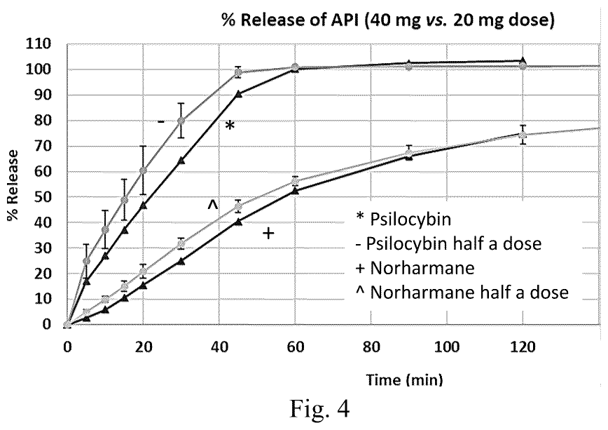

In addition, the release rate/profile of a patch comprising 20 mg drug load was compared with the release profile of half a dosage of the patch comprising 40 mg drug load, having the same Norharmane and Psilocybin ratio (3:1). In both patches, 100% of Psilocybin was released after 1 h and 75% and 82% of Norharmane was released after 2 h and 3 h, respectively (FIG. 4).

The patch release profile was examined after 9 months from the date of manufacturing. The patch retained its release profile with respect to the active agents, as compared to date of manufacturing.

1. A film comprising:
(i) a mucoadhesive polymer
(ii) a plasticizer
(iii) a first pharmaceutically active agent and a second pharmaceutically active agent, wherein:
the first pharmaceutically active agent is selected from psilocybin and a psilocybin derivative, including any salt and any combination thereof;
the second pharmaceutically active agent is selected from beta-carboline and a beta-carboline derivative, including any salt and any combination thereof;
a w/w concentration of the pharmaceutically active agent within the film is between 10 and 50%;
the first pharmaceutically active agent has at least 5 times greater water solubility at a temperature between 20 and 30° C. relative to the second pharmaceutically active agent; and wherein the film is characterized by a thickness between 50 and 500 um.

2. The film of claim 1, wherein a w/w ratio between the mucoadhesive polymer and the plasticizer within the film is between 10:1 and 1:1.

3. The film of claim 1, wherein said psylocibin derivative is represented by Formula 1: 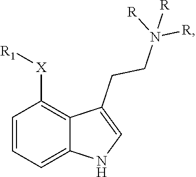
wherein X is O or OH; wherein R1 is H, POO⁻, POOH, or is absent; and wherein each R is independently hydrogen or methyl, with the proviso that the psylocibin derivative is not psylocibin.

4. The film of claim 1, wherein said psylocibin derivative is selected from norpsilocin, aeruginascin and psilocin, including any salt and any combination thereof.

5. The film of claim 1, wherein the first pharmaceutically active agent is characterized by a water solubility of at least 1 mg/ml at a temperature between 20 and 30° C.

6. The film of claim 1, wherein said beta-carboline derivative is represented by Formula 2: 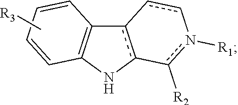
wherein R₁ is absent, or represents hydrogen or methyl; wherein R₂ is hydrogen or methyl; and wherein R₃ is absent or represents one or more substituents each independently selected from halogen, —OR′, —OH, —NO2, —CN, —CONH2, —CONR′2, —CNNR′2, —CSNR′2, —CONH—OH, —CONH—NH2, —NHCOR, —NHCSR, —NHCNR, —NC(═O)OR, —NC(═O)NR′, —NC(═S)OR′, —NC(═S)NR′, —SO2R′, —SOR′, —SR′, —SO2OR′, —SO2N(R′)2, —NHNR′2, —NNR′, —CO2H, —CO2R′, —OCOR, —OCOR′, —OC(═O)OR′, —OC(═O)NR′, —OC(═S)OR′, —OC(═S)NR′, or a combination thereof; wherein each R′ independently represents hydrogen, or methyl; and wherein a dashed line represents a single or a double bond, with the proviso that the beta-carboline derivative is not beta-carboline.

7. The film of claim 1, wherein said beta-carboline derivative comprises any one of Tryptoline, Pinoline, Harman, Harmine, Harmaline, Tetrahydroharmine, and 9-Methyl-β-carboline, or any combination thereof.

8. The film of claim 1, wherein said second pharmaceutically active agent is characterized by a water solubility of at most 1 mg/ml at a temperature between 20 and 30° C.

9. The film of claim 1, wherein psilocybin and beta-carboline, including any salt thereof are the sole pharmaceutically active agents within said film.

10. The film of claim 1, wherein an average molecular weight of said mucoadhesive polymer is between 10 KDa and 50 KDa; wherein said mucoadhesive polymer is or comprises polyvinyl alcohol (PVA); and wherein said plasticizer comprises glycerol.

11. (canceled)

12. (canceled)

13. The film of claim 1, wherein said film is further in contact with a non-adhesive layer comprising a polymer selected from: a polyether, a polyester, a polydioxanone, a polyphosphoester, a polyurethane, and a polyamide or any mixture or a co-polymer thereof.

14. The film of claim 1, wherein a release time of (i) said first pharmaceutically active agent, (ii) said second pharmaceutically active agent, or both (i) and (ii) from said film is at least 10 minutes.

15. The film of claim 1, wherein said film is in a form of a buccal patch suitable for application to a mucous tissue, and wherein said film is between 100 and 500 um thick; and wherein a weight concentration of the first pharmaceutically active agent within said buccal patch is between 10 and 25 mg.

16. (canceled)

17. A film comprising:
(i) a mucoadhesive polymer
(ii) a plasticizer
(iii) a first pharmaceutically active agent and a second pharmaceutically active agent, wherein:
the mucoadhesive polymer is a film forming polymer;
the first pharmaceutically active agent and the second pharmaceutically active agent are characterized by water solubility below 20 mg/ml at a temperature between 20 and 30° C., and are solid at a temperature between 20 and 30° C.;
a w/w concentration of the first pharmaceutically active agent and of the second pharmaceutically active agent within the film is between 10 and 50%;
the first pharmaceutically active agent has at least 5 times greater water solubility relative to the second pharmaceutically active agent; and wherein the film is characterized by a thickness between 50 and 500 um.

18. The film of claim 17, wherein a w/w ratio between the polymer and the plasticizer within the film is between 10:1 and 1:1; wherein the first pharmaceutically active agent is a psychoactive alkaloid; and wherein said second pharmaceutically active agent is characterized by a water solubility of at most 1 mg/ml at a temperature between 20 and 30° C.; optionally wherein the first pharmaceutically active agent comprises psilocybin, psilocybin derivative thereof, including any salt and any combination thereof; and wherein said second pharmaceutically active agent comprises beta-carboline, a beta-carboline derivative, including any salt or any combination thereof.

19. (canceled)

20. (canceled)

21. The film of claim 17, wherein the first pharmaceutically active agent is psylocibin, and wherein a weight concentration of psilocybin within said film is between 1 and 25 mg; and wherein said mucoadhesive polymer is PVA.

22. (canceled)

23. A method for preventing or treating a medical condition in a subject, comprising administering the film of claim 1 to the subject, thereby preventing or treating said medical condition.

24. The method of claim 23, wherein said administering comprising contacting the film with a biological tissue of said subject; and wherein said biological tissue comprises a mucous tissue, a dermal tissue, a muscle tissue, and a urinary bladder tissue or any combination thereof.

25. (canceled)

26. The method of claim 23, wherein said administering is selected from the group consisting of oral administration, nasal administration, and dermal administration, or any combination thereof; optionally wherein said oral administration comprises buccal administration, sublingual administration or both.

27. (canceled)

28. The method of claim 23, wherein said medical condition comprises an eating disorder or a neurodegenerative disease; optionally wherein said eating disorder comprises anorexia nervosa (AN).

29. (canceled)

30. (canceled)

31. (canceled)

32. (canceled)

33. (canceled)

34. (canceled)
